(19) United States
(12) Patent Application Publication
(10) Pub. No.: US 2004/0219237 A1
(43) Pub. Date: Nov. 4, 2004

(54) Process of using fermented Glycine max (L.) extract for enhancing natural killer cell activity and inhibitory activities to GABA and glutamate receptor binding

(76) Inventor: Kung-Ming Lu, Taipei (TW)

Correspondence Address: Leong C. Lei, PMB#1008, 1867 Ygnacio Valley Rd., Walnut Creek, CA, 94598, US

(21) Appl. No.: 10/853,220

(22) Filed: May 26, 2004

Related U.S. Application Data
(63) Continuation-in-part of application No. 10/178,364, filed on Jun. 25, 2002, now Pat. No. 6,733,801.

(51) Int. Cl. A61K 35/78; C12P 17/02

(52) U.S. Cl. 424/757; 435/41

(57) ABSTRACT

A process of using a fermented Glycine max (L.) extract comprising the steps of: mixing natural nutrient with medium solution to produce an agar medium; transferring various kinds of lactic acid bacteria and yeast into said agar medium and placing them into a thermostat for pure incubation; inoculating lactic acid bacteria and yeast and said agar medium to said soybean medium and incubating all bacteria in thermostat; separating said lactic acid bacteria and yeast from each other and transferring them into said organic soybean medium and starting acclimatized incubation; transferring all lactic acid bacteria and yeast from grouping incubation to said organic soybean medium and starting symbiotic incubation to form said fermented Glycine max (L.) extract, whereby the extract can be used for enhancing natural killer cell activity and inhibitory activities to GABA and glutamate receptor binding for prolonging sleeping time and reducing fatigue in cancer patients during chemotherapy was conducted.

### CROSS-REFERENCE:

This is a continuation-in-part application of the co-pending U.S. patent application Ser. No. 10/178,364.

### BACKGROUND OF THE INVENTION

1. Field of the Invention

This invention is related to a process of using a fermented Glycine max (L.) extract and in particular to one which can be used for enhancing natural killer cell activity and inhibitory activities to GABA and glutamate receptor binding.

2. Description of the Prior Art

The simple amino acids, GABA and glutamate, are the major inhibitory and excitatory neurotransmitters, respectively, in the human brain. Most, if not all, nerve cells in the brain have membrane receptors for extracellular GABA and glutamate. Highly specific transporters maintain low extracellular concentrations of these amino acids. These transporters also serve to remove synaptically released GABA and glutamate from the synaptic environment, thus rapidly terminating their synaptic actions.

GABA activates three different receptor classes such as GABA_{A}, GABA_{B} and GABA_{C} receptors. GABA_{A} receptors are ligand-gated chloride ion channels. These receptors are activated by GABA, muscimol and isoguvacine, and are inhibited by biscuculline, gabazine and (+)-β-hydrastine. The GABA_{A} receptors are of great importance as they play a pivotal role in the regulation of brain excitability, and many important drugs such as benzodiazepines, barbiturates, ethanol, neurosteroids, and some of the anticonvulsants and general anesthetics interact with these receptors.

Glutamate combines with two types of specific receptors, those that open an associated ion channel (ionotropic receptors) to allow the passage of small cations such as Na+ and Ca2+ into the cell. To date, three types of ionotropic receptors have been recognized and they are N-methyl-D-aspartate (NMDA), kainate and α-amino-3-hydroxy-5-methyl-4-isoxazole propionate (AMPA). Changes in glutamate transmission have been associated with a number of CNS pathologies; these include, acute stroke, chronic neurodegeneration, chronic pain, depression, drug dependency, epilepsy, Parkinson's disease and schizophrenia.

### SUMMARY OF THE INVENTION

This invention is related to a process of using a fermented Glycine max (L.) extract.

It is the primary object of the present invention to provide a process of using a fermented Glycine max (L.) extract which can be used for enhancing natural killer cell activity and inhibitory activities to GABA and glutamate receptor binding.

It is another object of the present invention to provide a concentrated fermentation broth which is a soymilk fermented with beneficial lactic acid bacteria and yeasts, wherein the microorganisms used in the manufacturing process included Lactobacillus paracasei, Lactobacillus burglarious, Saccharomyces cerivisiae, etc. that are often found as intestinal microflora and in some traditional fermentation products.

It is a further object of the present invention to provide a concentrated fermentation broth, which can be applied directly or formulated as capsules, tablets, or soluble powder.

To completely appreciate these and other objects of the present invention as well as the invention itself, all of which will become apparent to those skilled in the art, the following detailed description of the invention and the claims should be read in conjunction with the accompanying drawings.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a chart illustrating the effects of the present invention on locomotor activity in ICR mice;

FIG. 2 is a chart illustrating the effects of the present invention on pentobarbital-induced sleeping time;

FIG. 3 is a table illustrating the effect of the present invention on GABA_{A} receptor binding;

FIG. 4 is a table illustrating the effect of the present invention on glutamate receptor binding; and

FIG. 5 is a table illustrating the effect of the present invention on fatigue and appetite in cancer patients during chemotherapy.

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

The process of manufacturing a fermented Glycine max (L.) extract according to the present invention comprises the steps of:

1. Mixing the natural nutrient with medium solution to produce the special agar medium;

2. Transferring various kinds of lactic acid bacteria and yeast into the special agar medium produced in Step 1 individually and placing them into the thermostat for pure incubation under the controlled condition;

3. Removing the fat contained in the soybeans, adding distilled water and boiling for about 2 hours to get the soybean solution, filtering the soybean solution again to get the organic soybean medium;

4. Inoculating lactic acid bacteria and yeast and the medium originated from the pure culture prepared in Step 2 to the soybean medium prepared in Step 3 and incubating all bacteria in thermostat under the controlled condition;

5. Separating the lactic acid bacteria and yeast from each other according to the groupings and transferring them into the organic soybean medium prepared in Step 3 by groupings and starting the acclimatized incubation;

6. Transferring all lactic acid bacteria and yeast from grouping incubation of prepared in Step 5 to the organic soybean medium prepared in Step 3 and starting the symbiotic incubation;

7. Heating the microorganism culture prepared in Step 6 at high temperature to stop the growth of original bacteria and removing the microorganisms in the medium and condensing the filtered solution to 1/15 (remove 93-96% moisture); and

8. Placing the concentrated solution prepared in Step 7 at room temperature for at least 2 months so that the concentrate shall separate and the upper layer is collected into the container and heat is applied to get the end product.

In the manufacturing procedures, the chemistry, manufacturing, and controls were achieved by the different assays including genistein and daidzein contents, HPLC fingerprints, pH values, and physical properties (odors, appearances, colors, clarity, tastes and precipitates).

### In Vitro Study on Binding Activities of GABA and Glutamate Receptors

The effect of the concentrated fermentation broth according to the present invention on GABA receptor was evaluated with radioligand binding assay. The assay measured the binding activity of [3H] Muscimol to the agonist site of the GABA_{A} receptors. Whole brain (cerebellum) membranes of male Wistar rats weighing 175±25 g were prepared in Tris-HCl pH 7.4 buffer using standard techniques 2˜4. A 10 mg aliquot of membrane was incubated with 1 nM [³H]Muscimol for 10 minutes at 4° C. Non-specific binding was estimated in the presence of 100 nM muscimol. Membranes were filtered and washed 3 times and the filters were counted to determine [³H] Muscimol specifically bound.

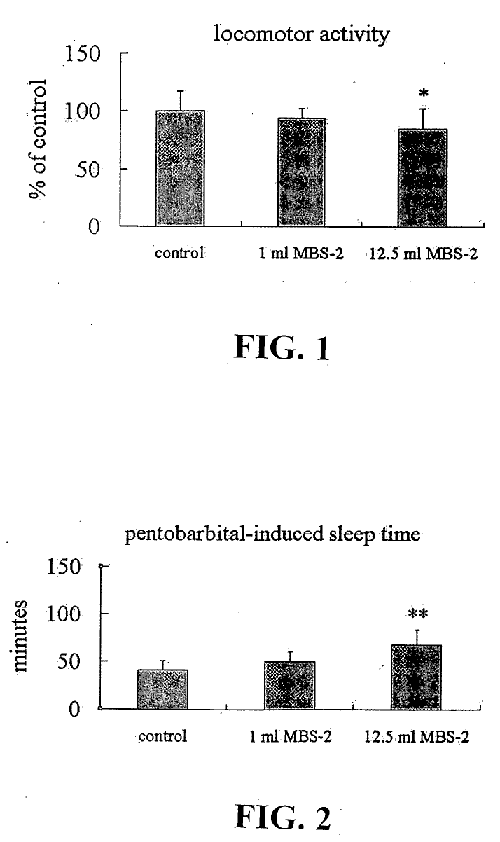

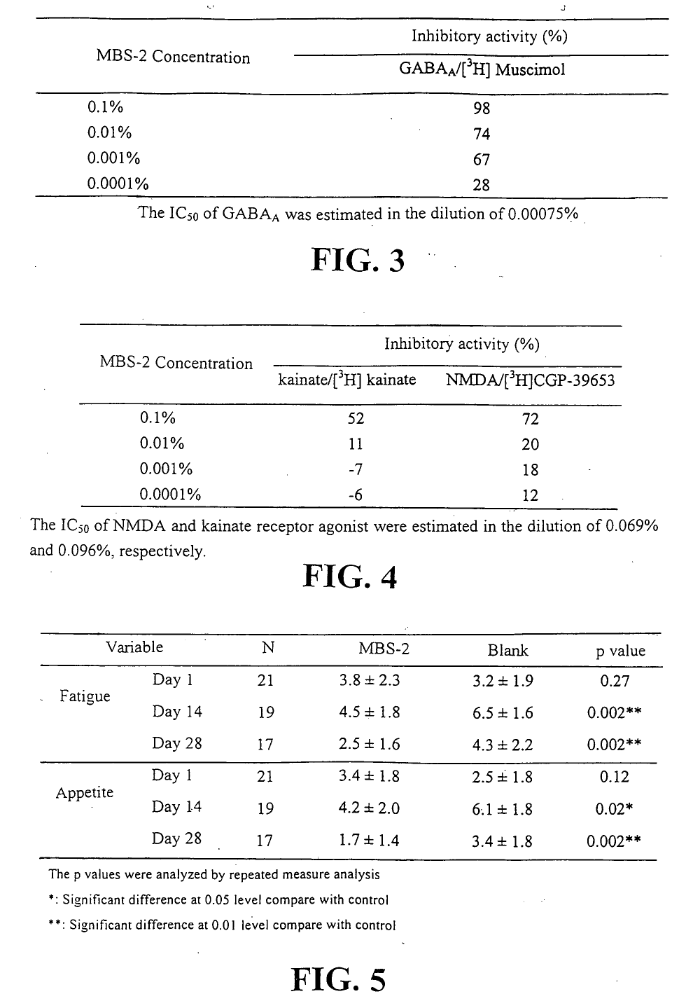

In the radiolabled-binding assay described above, the concentrated fermentation broth according to the present invention was screened at different folds of dilution. The IC₅₀ of product was calculated in the dilution of 0.00075% (FIG. 1). This was shown that the concentrated fermentation broth has the strong activity on GABA agonist site.

The effect of the concentrated fermentation broth according to the present invention on glutamate receptor was evaluated with radioligand binding assay. The assay measured the binding of CGP-39653, a selective antagonist, to the agnoist site of the NMDA receptor. A membranes preparation from male rat brain cortex is isolated by standard techniques. Twenty milligrams of membrane preparation was incubated with 2.0 nM[³H] CGP-39653 for 20 minutes at 4° C. Non-specific binding was estimated in the presence of 1,000 μM L-glutamate. Membranes were filtered and washed 3 times and the filters are counted to determine [³H] CGP-39653 bound. Similar protocol measured the binding of glutamate, kainate receptor which using 5.0 nM [³H] kainate as a ligand. The solution according to the present invention was screened at different folds of dilution. The IC₅₀ of NMDA and kainate receptor agonist were estimated in the dilution of 0.069% and 0.096%, respectively (FIG. 4).

The effects of administrating the concentrated fermentation broth according to the present invention on locomotor activity and pentobarbital-induced sleeping time were studied in ICR mice. The oral administration of the concentrated fermentation broth according to the present invention at doses equivalent to human use 13 ml significantly reduced locomotor activity and prolonged pentobarbital-induced sleeping time in mice (FIGS. 1 and 2, p<0.05).

An 8-week, open-label, randomized, cross-over, and comparative pilot clinical study for the evaluation of the impact of concentrated fermentation broth for reducing fatigue in cancer patients during chemotherapy was conducted. The data have shown the intensity of fatigue decreased significantly from day 14 to day 28 and appetite was highly improved after administration of the concentrated fermentation broth for cancer patients by the 10-cm visual analog scale (VAS) measurement (p<0.05; FIG. 5).

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A process of using a fermented Glycine max (L.) extract comprising the steps of:
a. mixing natural nutrient with medium solution to produce an agar medium;
b. transferring various kinds of lactic acid bacteria and yeast into said agar medium produced individually and placing them into a thermostat for pure incubation under controlled condition;
c. inoculating lactic acid bacteria and yeast and said agar medium to said soybean medium and incubating all bacteria in thermostat under controlled condition;
d. separating said lactic acid bacteria and yeast from each other according to groupings and transferring them into said organic soybean medium and starting acclimatized incubation;
e. transferring all lactic acid bacteria and yeast from grouping incubation to said organic soybean medium and starting symbiotic incubation to form said fermented Glycine max (L.) extract; and
f. administering said extract in a predetermined amount to a human subject in need thereof in an amount effective to enhance natural killer cell activity and inhibitory activities to GABA and glutamate receptor binding for prolonging pentobarbital-induced sleeping time and reducing fatigue in cancer patients during chemotherapy was conducted.

2. The process of using a fermented Glycine max (L.) extract as claimed in claim 1, wherein at least one lactic acid bacteria and one yeast are transferred into said agar medium.

3. The process of using a fermented Glycine max (L.) extract as claimed in claim 1, wherein said extract is capable of enhancing inhibitory activities to GABA and glutamate receptor binding.

4. The process of using a fermented Glycine max (L.) extract as claimed in claim 1, wherein said extract is capable of prolonging sleeping time.

5. The process of using a fermented Glycine max (L.) extract as claimed in claim 1, wherein said extract is capable of reducing fatigue in cancer patients during chemotherapy was conducted.

6. The fermented Glycine max (L.) extract as claimed in claim 1, wherein said extract can be applied directly or formulated as capsules, tablets, or soluble powder.
